(19) United States
(12) Patent Application Publication
(10) Pub. No.: US 2025/0281615 A1
(43) Pub. Date: Sep. 11, 2025

(54) SYSTEMS AND METHODS FOR TREATING CANCERS OF THE SKIN

(71) Applicant: Bruce Philip ROBINSON, New York, NY (US)

(72) Inventor: Bruce Philip ROBINSON, New York, NY (US)

(21) Appl. No.: 19/072,698

(22) Filed: Mar. 6, 2025

Related U.S. Application Data
(60) Provisional application No. 63/562,002, filed on Mar. 6, 2024.
(60) Provisional application No. 63/716,066, filed on Nov. 4, 2024.

(51) Int. Cl. A61K 41/00; A61B 18/00; A61B 18/14; A61B 18/20; A61K 9/00; A61K 31/5377; A61P 35/00

(52) U.S. Cl. CPC A61K 41/0028; A61B 18/1402; A61B 18/203; A61K 9/0014; A61K 31/5377; A61P 35/00; A61B 2018/0047; A61B 2018/00601

(57) ABSTRACT

A method for treating squamous cell carcinomas (SCCs), basal cell carcinomas (BCCs), or other cancers of the skin may include administering a plurality of laser pulses to a cancerous lesion using one or more lasers and/or using radio frequency therapy, and, optionally, for a predetermined treatment period, repeatedly applying a topical medication for treating pre-cancerous or cancerous lesions to the cancerous lesion.

### CROSS-REFERENCE TO RELATED APPLICATIONS

This application claims the benefit of U.S. Provisional Application No. 63/562,002, filed Mar. 6, 2024, and U.S. Provisional Application No. 63/716,066, filed Nov. 4, 2024, the entire contents of each of which are incorporated herein by reference.

### FIELD

The present disclosure relates generally to treating cancers of the skin.

### BACKGROUND

Basal cell carcinomas (BCC) and squamous cell carcinomas (SCC) are the most common forms of cancer in the United States, typically arising from excessive, long-term exposure to ultraviolet radiation. The depth of penetration of these skin cancers can vary. The early forms are SCCs in situ and superficial BCCs, which inhabit the epidermis and can extend to its full thickness. These cancers differ in their origins as SCCs in situ begin in the upper portion of the epidermis, while superficial BCCs begin in the lower portion at the basal layer. Nodular BCCs extend past the epidermis and into the dermis. The current most commonly accepted treatments for SCCs and BCCs, depending on the location and depth, include surgical removal, electrodessication and curettage, or Mohs surgery. Although these treatments were created to limit the destruction of surrounding healthy cells, all treatments typically leave a scar.

### SUMMARY

Provided are systems and methods for treating cancers of the skin that employ fractional laser and/or radio frequency therapy, optionally in combination with topical medications. In the provided systems and methods, a cancerous lesion of the skin, such an SCC or a BCC, is treated via bulk heating of the skin at the site of the SCC or BCC or other cancer of the skin using fractional laser and/or radio frequency energy therapy. Optionally, fractional photothermolysis is used in addition to bulk heating to create small wounds in the skin at the site of the SCC, a BCC, or another cancer of the skin. Optionally, subsequently, topical medication typically used for treating pre-cancerous lesions and/or topical medication used for treating cancerous lesions is applied to the site of the SCC, BCC, or other cancer of the skin over the course of a predefined treatment period. The fractional laser and/or radio frequency therapy may maximize the depth of skin penetration of the topical medication, resulting in greater skin cancer and/or other cancer of the skin destruction and reducing the likelihood of cancer recurrence.

The disclosed systems and methods may meet the needs of a wide range of patients. Since the provided techniques are non-invasive, patients on anticoagulants can be treated without having to discontinue their medications as they would be required to do for an invasive procedure to avoid blood clots, stroke, and heart attack. Additionally, the provided systems and methods are beneficial to elderly patients whose thin skin often requires complex closures, skin grafts, or flaps, as well as prolonged wound care after invasive procedures. Furthermore, the provided systems and methods may be useful for patients who prefer to avoid extensive downtime or limitations to their mobility as well as those who are concerned about scarring, such as those who are physically active or in the public eye.

### BRIEF DESCRIPTION OF THE FIGURES

The invention will now be described, by way of example only, with reference to the accompanying drawings, in which:

FIG. 1 shows a system for treating cancers of the skin, according to some embodiments.

FIG. 2 shows a method for treating cancers of the skin, according to some embodiments.

FIG. 3 shows an exemplary treatment system with at least one sensor, according to some embodiments.

FIG. 4A shows a photograph of a biopsy-proven squamous cell carcinoma prior to treatment with fractional laser therapy.

FIG. 4B shows a photograph of a biopsy-proven squamous cell carcinoma immediately following treatment with fractional laser therapy.

FIG. 4C shows a photograph of a biopsy-proven superficial basal cell carcinoma prior to treatment with fractional laser therapy.

FIG. 4D shows a photograph of a biopsy-proven superficial basal cell carcinoma following treatment with fractional laser therapy and tirbanibulin ointment application.

FIG. 5 is an exemplary computing system.

### DETAILED DESCRIPTION

The provided systems and methods for treating squamous cell carcinomas, basal cell carcinomas, and other cancers of the skin utilize a combination of fractional laser, radio frequency therapy, and/or topical medications. In various embodiments, fractional photothermolysis and/or radio frequency therapy is used to create small wounds in the skin at the site of a SCC, a BCC, or other cancer of the skin. Subsequently, topical medication for treating pre-cancerous lesions may be applied to the site of the SCC, BCC, or other cancer of the skin over the course of a predefined treatment period. The fractional laser and/or radio frequency therapy may maximize the depth of skin penetration of the topical medication, resulting in greater skin cancer destruction and reducing the likelihood of cancer recurrence. The fractional laser and/or radio frequency therapy may additionally, or alternatively, cause bulk heating and subsequent release of anti-tumor chemicals in the skin, such as heat shock proteins (HSP) (such as HSP 90), which may induce proinflammatory cytokines ILG, IL12, IL15, TNP, and nitric oxide, and may increase the accumulation of the tumor suppressor p53 resulting in destruction and subsequent resolution of the skin cancer. Such systems and methods provide effective treatment of skin cancers in a non-invasive, low-cost procedure.

In the following description, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes,” “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating,” or the like refer to the action and processes of a computer system, or similar electronic computing device, that manipulates and transforms data represented as physical (electronic) quantities within the computer system memories or registers or other such information storage, transmission, or display devices.

The present disclosure in some aspects also relates to devices or systems for performing the operations herein. The devices or systems may be specially constructed for the required purposes, may comprise a general-purpose computer selectively activated or reconfigured by a computer program stored in the computer, or may include any combination thereof. Computer instructions for performing the operations herein can be stored in any combination of non transitory, computer-readable storage medium, such as, but not limited to, any type of disk, including USB flash drives, external hard drives, optical disks, CD-ROMs, magnetic-optical disks, read-only memories (ROMs), random access memories (RAMs), EPROMS, EEPROMs, magnetic or optical cards, or any type of media suitable for storing electronic instructions, and each coupled to a computer system bus. One or more instructions for performing the operations herein may be implemented in or executed by one or more Application Specific Integrated Circuits (ASICs), Field Programmable Gate Arrays (FPGAs), Digital Signal Processing units (DSPs), Graphics Processing Units (GPUs), Central Processing Units (CPUs), or any other suitable processing unit. Furthermore, the computers referred to herein may include a single processor or may be architectures employing multiple processor designs for increased computing capability.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, the present invention is not described with reference to any particular programming language. It will be appreciated that a variety of programming languages may be used to implement the teachings of the present invention as described herein.

### I. Definitions

The terms “baseline” or “baseline value” used interchangeably herein can refer to a measurement or characterization of a symptom before the administration of the therapy (e.g., a topical ointment as described herein and/or an energy delivery device as described herein) or at the beginning of administration of the therapy. The baseline value can be compared to a reference value in order to determine the reduction or improvement of a symptom of a disease, such as a cancer. The terms “reference” or “reference value” used interchangeably herein can refer to a measurement or characterization of a symptom after administration of the therapy (e.g., a topical ointment as described herein and/or an energy delivery device as described herein). The reference value can be measured one or more times during a dosage regimen or treatment cycle or at the completion of the dosage regimen or treatment cycle. A “reference value” can be an absolute value; a relative value; a value that has an upper and/or lower limit; a range of values; an average value; a median value: a mean value; or a value as compared to a baseline value.

Similarly, a “baseline value” can be an absolute value; a relative value; a value that has an upper and/or lower limit; a range of values; an average value; a median value; a mean value; or a value as compared to a reference value. The reference value and/or baseline value can be obtained from one individual, from two different individuals or from a group of individuals (e.g., a group of two, three, four, five or more individuals).

As used herein, “complete response” or “CR” refers to disappearance of all target lesions; “partial response” or “PR” refers to a significant decrease in the size of target lesions, taking as reference the baseline size.

As used herein, “progression free survival” or “PFS” refers to the length of time during and after treatment during which the disease being treated (e.g., skin cancer) does not get worse. Progression-free survival may include the amount of time patients have experienced a complete response or a partial response, as well as the amount of time patients have experienced stable disease.

As used herein, “objective response rate” or “ORR” refers to the sum of complete response (CR) rate and partial response (PR) rate.

As used herein, “overall survival” or “OS” refers to the percentage of individuals in a group who are alive after a particular duration of time.

The term “patient” or “subject” includes human and other mammalian subjects such as non-human primates, rabbits, rats, mice, and the like and transgenic species thereof, that receive either prophylactic or therapeutic treatment.

The term “effective amount,” in the context of treatment of a solid tumor by administration of a topical ointment as described herein, refers to an amount of such topical ointment that is sufficient to inhibit the occurrence or ameliorate one or more symptoms of a solid tumor.

The term “pharmaceutically acceptable” means approved or approvable by a regulatory agency of the Federal or a state government or listed in the U.S. Pharmacopeia or other generally recognized pharmacopeia for use in animals, and more particularly in humans. The term “pharmaceutically compatible ingredient” refers to a pharmaceutically acceptable diluent, adjuvant, excipient, or vehicle with which the topical ointment is formulated.

The phrase “pharmaceutically acceptable salt,” refers to pharmaceutically acceptable organic or inorganic salts. Exemplary salts include sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p toluenesulfonate, and pamoate (i.e., 1,1′-methylene bis-(2 hydroxy-3-naphthoate) salts. A pharmaceutically acceptable salt may further comprise an additional molecule such as, e.g., an acetate ion, a succinate ion or other counterion. A counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

The use of the alternative (e.g., “or”) should be understood to mean either one, both, or any combination thereof of the alternatives. As used herein, the indefinite articles “a” or “an” should be understood to refer to “one or more” of any recited or enumerated component.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

The terms “treatment” or “treat” refer to slowing, stopping, or reversing the progression of the disease or condition in a patient, as evidenced by a decrease or elimination of a clinical or diagnostic symptom of the disease or condition. Treatment can include, for example, a decrease in the severity of a symptom, the number of symptoms, or frequency of relapse.

As used herein, the term “synergy” or “synergistic effect” when used in connection with a description of the efficacy of a combination of agents, means any measured effect of the combination which is greater than the effect predicted from a sum of the effects of the individual agents.

As used herein, the term “additive” or “additive effect” when used in connection with a description of the efficacy of a combination of agents, means any measured effect of the combination which is similar to the effect predicted from a sum of the effects of the individual agents.

The terms “once about every week,” “once about every two weeks,” or any other similar dosing interval terms as used herein mean approximate numbers. “Once about every week” can include every seven days±one day, i.e., every six days to every eight days. “Once about every two weeks” can include every fourteen days±two days, i.e., every twelve days to every sixteen days. “Once about every three weeks” can include every twenty-one days±three days, i.e., every eighteen days to every twenty-four days. Similar approximations apply, for example, to once about every four weeks, once about every five weeks, once about every six weeks, and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

As described herein, any concentration range, percentage range, ratio range, or integer range is to be understood to include the value of any integer within the recited range and, when appropriate, fractions thereof (such as one tenth and one hundredth of an integer), unless otherwise indicated.

### II. Systems and Methods For Treating Cancers Of The Skin

The various aspects and embodiments described in this section in the context of systems for treating cancers of the skin also apply to methods of treatment of skin cancer. Such methods also apply to a topical medication for use according to the methods described herein, and/or energy from an energy delivery device for use according to the methods described herein, unless indicated otherwise. The following section describes various aspects of dosing and treatment regimens using the systems described below, any and all of which apply to the methods and/or uses described herein.

In some embodiments, provided herein is a method of treating skin cancer in an individual, the method comprising administering to a cancerous lesion on the individual a topical ointment in combination with energy from an energy delivery device. Alternatively, in some embodiments, provided herein is a method of treating skin cancer in an individual, the method comprising administering to a cancerous lesion on the individual energy from an energy delivery device in combination with a topical ointment.

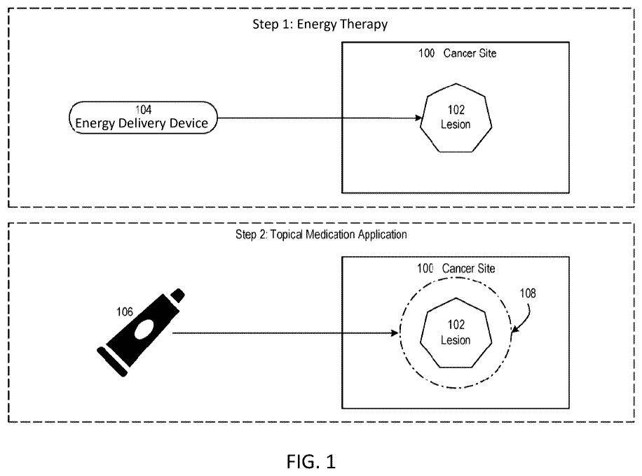

FIG. 1 shows an exemplary system for treating squamous cell carcinomas, basal cell carcinomas, and other cancers of the skin. The system may include one or more energy delivery devices 104 and a topical medication 106. During a first stage of the treatment process, energy delivery device(s) 104 may be used to treat a cancerous lesion 102 (e.g., a squamous cell carcinoma, basal cell carcinoma, or other cancer of the skin). In some embodiments, the energy delivery device(s) 104 delivers energy to a cancerous lesion 102 to heat the cancerous lesion 102 using a fractional laser and/or radio frequency therapy technique. The energy delivery device(s) 104 may include, for example, at least one laser, at least one radio frequency energy emitting device, and/or at least one other type of energy radiating device that may sufficiently penetrate and at least partially destroy at least a portion of a cancerous lesion. The fractional laser and/or radio frequency therapy may additionally, or alternatively, cause bulk heating and subsequent release of anti-tumor chemicals in the skin, such as heat shock proteins (HSP) (such as HSP 90), which may induce proinflammatory cytokines ILG, IL12, IL15, TNP, and nitric oxide, and may increase the accumulation of the tumor suppressor p53. Following the energy delivery treatment, during a second stage of the treatment process, topical medication 106 may be repeatedly applied to lesion 102 over the course of a predetermined treatment period.

The site 100 of cancerous legion 102 may be on the patient's skin at any location. Potential locations of cancerous lesion 102 include (but are not limited to) a forehead, an earlobe, a scalp, a nose, a zygomatic arch, a costal margin, a back, a flank, an arm, a foot, a hand, or a leg.

Energy delivery device(s) 104 can include at least one, at least two, at least three, at least four, or at least five different lasers. For example, energy delivery device(s) 104 can include a first laser and a second laser. The first laser may have higher or lower energy than the second laser. During a fractional laser therapy treatment, the first laser may administer microscopic treatment zones (MTZs) over multiple passes to a cancer site 100 on the patient's skin that includes cancerous lesion 102. MTZs may then be administered over multiple passes to cancer site 100 by the second laser.

Alternatively, energy delivery device(s) 104 may be a single laser with multiple settings, for example a single laser with at least one, at least two, at least three, at least four, or at least five settings. For instance, energy delivery device(s) 104 may be a single laser with a first laser setting and a second laser setting. The first laser setting may have higher or lower energy than the second laser setting. During the fractional laser therapy treatment, the first laser setting may be used to administer microscopic treatment zones (MTZs) over multiple passes to a cancer site 100 on the patient's skin that includes cancerous lesion 102. MTZs may then be administered over multiple passes to cancer site 100 using the second laser setting.

Any suitable laser(s) can be used. In some embodiments, one or more lasers are used that generate light have a wavelength in a range from 1450 nm to 10,600 nm. Any suitable laser pulse energy may be used. In some embodiments, pulse energies are used that are up to 100 mJ.

In some embodiments, one or more lasers are used that generate laser light having a wavelength in a range from 1000 nm to 10,600 nm, which may provide a number of unexpected advantages for treating basal cell and squamous cell carcinomas. Treatment with laser pulses having wavelengths within this range may provide effective penetration into the skin, reaching the deeper layers where basal cell and squamous cell carcinomas reside. This may ensure that the laser energy can directly target and destroy tumor cells, while minimizing impact on the superficial layers of skin. Treatment with laser pulses having wavelengths within this range may produce a controlled thermal effect, which may raise tissue temperatures above 42° C. Such elevated temperatures and resulting bulk heating (including tissue surrounding the tissue that directly receives laser treatment) may denature proteins and destabilize malignant cells, leading to their destruction. Additionally, the thermal effect may induce a heat shock response, which may include elevated levels of Heat Shock Protein 90 (HSP90). Without being bound by any theory, while HSP90 may help stabilize normal cellular processes, its impact on malignant cells can lead to the denaturing of tumor-specific proteins necessary for their survival. The stress and damage induced by these wavelengths can lead to the accumulation of tumor suppressor gene products, such as p53. This can trigger apoptosis (cell death) pathways, further aiding in the elimination of cancerous cells. Treatment with laser pulses having wavelengths within this range can elicit a pro-inflammatory response, with the release of cytokines like IL-6, IL-12, IL-15, TNF, and nitric oxide. These molecules help recruit immune cells to the site, enhancing the body's natural ability to combat and eradicate cancer cells. Treatment with laser pulses having wavelengths within this range may create microthermal zones that increase in depth and width with higher energies. These zones may destroy cancerous tissue while sparing healthy surrounding tissue, which can aid in recovery and reduce scarring. Treatment with laser pulses having wavelengths within this range may induce photothermal effects, where light energy is converted into heat, and photomechanical effects, which can cause physical disruption of cellular structures, both contributing to tumor destruction. As such, treatment with laser pulses having wavelengths within this range for the treatment of basal cell and squamous cell carcinomas may maximize the therapeutic effect while minimizing damage to healthy tissues, leveraging both direct tumor destruction and immune-mediated tumor suppression.

In some embodiments, a first laser of multiple lasers may have a wavelength between 1450 nm and 1650 nm, between 1475 nm and 1625 nm, between 1500 nm and 1600 nm, or between 1525 nm and 1575 nm. For example, the first laser may have a wavelength of approximately 1530 nm, 1535 nm, 1540 nm, 1545 nm, 1550 nm, 1555 nm, or 1560 nm. The pulse energy of the first laser may be between 1 and 100 mJ, between 35 and 75 mJ, between 40 and 70 mJ, between 45 and 65 mJ, or between 50 and 60 mJ, for instance about 51 mJ, 52 mJ, 53 mJ, 54 mJ, 55 mJ, 56 mJ, 57 mJ, 58 mJ, 59 mJ, or 60 mJ. A second laser of the multiple lasers may have a wavelength between 1800 nm and 2000 nm, between 1825 nm and 1975 nm, between 1850 and 1950 nm, between 1875 and 1950 nm, or between 1900 and 1950 nm. For example, the second laser may have a wavelength of approximately 1923 nm, 1925 nm, 1927 nm, or 1929 nm. The pulse energy of the second laser may be between 1 and 100 mJ, between 1 and 15 mJ, between 2 and 14 mJ, between 3 and 13 mJ, between 4 and 12 mJ, or between 5 and 11 mJ, for instance about 6 mJ, 7 mJ, 8 mJ, 9 mJ, or 10 mJ. In some embodiments, the first laser is an erbium laser and the second laser is a thulium laser. The first laser may administer MTZs over at least 5, at least 6, at least 7, at least 8, at least 9, at least 10, at least 11, at least 12, at least 13, at least 14, or at least 15 passes. The first laser may administer MTZs over 1-15 passes. Similarly, the second laser may administer MTZs over at least 5, at least 6, at least 7, at least 8, at least 9, at least 10, at least 11, at least 12, at least 13, at least 14, or at least 15 passes. Between subsequent laser passes, the interval time may be less than 5 seconds, 4 seconds, 3 seconds, two seconds, or 1 second to limit the ability of heat to diffuse through the epidermis, dermis, and through blood perfusion.

In some embodiments, energy delivery device(s) 104 is a single laser with multiple settings, for example a single laser with at least one, at least two, at least three, at least four, or at least five settings. For instance, energy delivery device(s) 104 may be a single laser with a first laser setting and a second laser setting. The first laser setting may have higher or lower energy than the second laser setting. During the fractional laser therapy treatment, the first laser setting may be used to administer microscopic treatment zones (MTZs) over multiple passes to a cancer site 100 on the patient's skin that includes cancerous lesion 102. MTZs may then be administered over multiple passes to cancer site 100 using the second laser setting.

The first laser setting may have a wavelength between 1450 nm and 1650 nm, between 1475 nm and 1625 nm, between 1500 nm and 1600 nm, or between 1525 nm and 1575 nm. For example, the first laser setting may have a wavelength of approximately 1530 nm, 1535 nm, 1540 nm, 1545 nm, 1550 nm, 1555 nm, or 1560 nm. The pulse energy of the first laser setting may be between 35 and 75 mJ, between 40 and 70 mJ, between 45 and 65 mJ, or between 50 and 60 mJ, for instance about 51 mJ, 52 mJ, 53 mJ, 54 mJ, 55 mJ, 56 mJ, 57 mJ, 58 mJ, 59 mJ, or 60 mJ. The second laser setting may have a wavelength between 1800 nm and 2000 nm, between 1825 nm and 1975 nm, between 1850 and 1950 nm, between 1875 and 1950 nm, or between 1900 and 1950 nm. For example, the second laser setting may have a wavelength of approximately 1923 nm, 1925 nm, 1927 nm, or 1929 nm. The pulse energy of the second laser setting may be between 1 and 20 mJ, between 2 and 14 mJ, between 3 and 13 mJ, between 4 and 12 mJ, or between 5 and 11 mJ, for instance about 6 mJ, 7 mJ, 8 mJ, 9 mJ, or 10 mJ. In some embodiments, the first laser setting is an erbium laser and the second laser is a thulium laser. The first laser setting may be used to administer MTZs over at least 5, at least 6, at least 7, at least 8, at least 9, at least 10, at least 11, at least 12, at least 13, at least 14, or at least 15 passes. The first laser setting may be used to administer MTZs over 1-15 passes. Similarly, the second laser setting may be used to administer MTZs over at least 5, at least 6, at least 7, at least 8, at least 9, at least 10, at least 11, at least 12, at least 13, at least 14, or at least 15 passes. Between subsequent laser passes, the interval time may be less than 5 seconds, 4 seconds, 3 seconds, two seconds, or 1 second to limit the ability of heat to diffuse through the epidermis, dermis, and through blood perfusion.

In some embodiments, energy delivery device(s) 104 can include at least one radio frequency energy emitting device. The at least one radio frequency energy emitting devices may be configured to apply electrical energy to a treatment site in which the electrical energy has a frequency in the radio frequency range. The at least one radio frequency energy emitting devices may be configured to provide different amounts of energy, which may be user controlled and/or automatically controlled. For example, energy delivery device(s) 104 can include a radio frequency energy emitting device that can be controlled to provide higher energy or lower energy (e.g., higher/lower frequency and/or higher/lower amplitude). Optionally, the energy delivery device(s) 104 can include multiple radio frequency energy emitting devices that may each be configured to deliver a different level of radio frequency energy.

During a radio frequency therapy treatment, the radio frequency energy emitting device(s) may administer bulk heating over one or more passes at a first energy level to a cancer site 100 on the patient's skin that includes cancerous lesion 102. Bulk heating may then be administered over one or more passes at a second energy level to cancer site 100. This may be done using different radio frequency energy emitting devices. Alternatively, energy delivery device(s) 104 may include a single radio frequency energy emitting device having multiple radio frequency settings, for example a single radio frequency emitting device with at least one, at least two, at least three, at least four, or at least five radio frequency settings. In embodiments having multiple radio frequency energy emitting devices, a first radio frequency energy emitting device may be configured to provide higher energy or lower energy than a second radio frequency energy emitting device setting. During radio frequency energy treatment, the first radio frequency energy emitting device may be used to administer bulk heating over one or more passes to a cancer site 100 on the patient's skin that includes cancerous lesion 102. The second radio frequency energy emitting device may be used to administer bulk heating over one or more passes to cancer site 100. Thus, the cancerous lesion 102 may be treated with one or more radio frequency energy levels over one or more passes. In embodiments that utilize different radio frequency energy levels, each radio frequency energy level may be applied in one or more passes, which may be the same number of passes for each energy level or a different number of passes for different radio frequency energy levels. For example, a first radio frequency energy level may be applied over multiple passes and a second radio frequency energy level may be applied over a single pass. A time interval between passes may be 1-20 seconds. A time interval between passes may be less than 5 seconds, 4 seconds, 3 seconds, two seconds, or 1 second to limit the ability of heat to diffuse through the epidermis, dermis, and through blood perfusion. A first radio frequency energy emitting device may administer bulk heating over 1-50 passes. Optionally, a first radio frequency energy emitting device may administer bulk heating over at least 5, at least 6, at least 7, at least 8, at least 9, at least 10, at least 11, at least 12, at least 13, at least 14, at least 15 passes, at least 20 passes, at least 50 passes, etc. Similarly, the second radio frequency emitting device may administer bulk heating over 1-50 passes. Optionally, the second radio frequency emitting device may administer bulk heating over at least 5, at least 6, at least 7, at least 8, at least 9, at least 10, at least 11, at least 12, at least 13, at least 14, at least 15 passes, at least 20 passes, at least 50 passes, etc.

Optionally, laser and radio frequency energy can be used in combination in the same or separate devices. For example, one or more first passes may include applying laser energy to the cancerous lesion 102 and one or more second passes may include applying radio frequency energy to the cancerous lesion 102 or vice versa.

Topical medication 106 may be or comprise an ointment or other topical drug. The topical medication 106 may comprise an ointment or other topical drug conventionally used for treating pre-cancerous lesions and/or an ointment or other drug conventionally used for treating cancerous lesions, including any pharmaceutically acceptable salts thereof. For example, topical medication may be or comprise a medication for treating actinic keratoses such as tirbanibulin (brand name Klisyri) or imiquimod (brand name Aldara). When used in combination with fractional laser and/or radio frequency therapy, such topical medications can penetrate deeper into the skin and, as a result, may effectively inhibit the spread or cause destruction of cancerous cells at skin depths up to (and possibly exceeding) 2000 μm. Alternatively, topical medication 106 can be a topical chemotherapy agent, for example 5-fluoroacil. Topical medication 106 may be applied by the patient.

The treatment period over which topical medication 106 is applied following fractional laser and/or radio frequency therapy may vary based on the characteristics of lesion 102 (e.g., the type of lesion, the nature of the lesion (flat or raised), etc.). In some embodiments, the treatment period may be at least 1 day, at least 2 days, at least 3 days, at least 4 days, at least 5 days, at least 6 days, at least 7 days, at least 8 days, at least 9 days, or at least 10 days. In other embodiments, the treatment period may be at least 1 month, at least 2 months, at least 6 months, or at least 1 year. Topical medication 106 may be applied repeatedly (e.g., periodically) during the treatment period. For example, topical medication 106 may be applied once per day, twice per day, three times per day, four times per day, or five times per day during the treatment period. To create a field effect of pre-cancerous cell destruction, topical medication 106 may be applied to both lesion 102 and a region 108 that surrounds lesion 102. Region 108 may be an approximately circular region with a diameter that extends at least 5 mm, at least 10 mm, at least 15 mm, at least 20 mm, at least 25 mm, at least 30 mm, at least 35 mm, at least 40 mm, at least 45 mm, or at least 50 mm past a border of lesion 102.

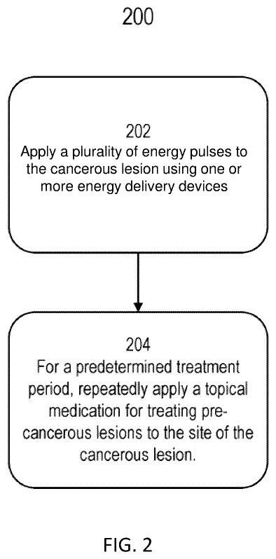

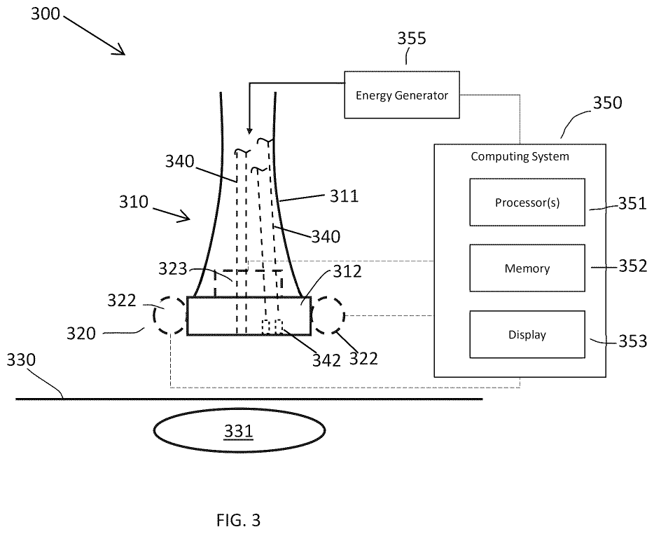

FIG. 2 shows an exemplary method 200 for treating squamous cell carcinomas, basal cell carcinomas, and other cancers of the skin. Method 200 may be performed using a system for treating squamous cell carcinomas, basal cell carcinomas, and/or other cancers of the skin such as the system illustrated in FIG. 1 and/or system 300 of FIG. 3. After a cancerous lesion has been identified (e.g., via biopsy), at step 202, a plurality of energy pulses (e.g., laser and/or radio frequency pulses) may be applied to the lesion using one or more energy delivery device(s) (e.g., energy delivery device(s) 104 shown in FIG. 1). The energy pulses may be administered to the lesion over sets of multiple passes. Subsequently, at step 204, a topical medication for treating pre-cancerous lesions (e.g., topical medication 106 shown in FIG. 1) may be repeatedly applied to the cancerous lesion over the course of a predetermined treatment period.

In some embodiments, prior to step 202, the treatment site may be numbed (e.g., using lidocaine with epinephrine). This may enhance bulk heating of the treatment site by the energy pulses. The interval time between passes of the laser(s) and/or radio frequency emitting device(s) may be limited (e.g., to less than 5 seconds) to limit the ability of heat to diffuse through the epidermis, dermis, and through blood perfusion. When the topical medication is applied (step 204), it may be applied to both the lesion itself and to a region surrounding the lesion to create a field effect of pre-cancerous cell destruction.

FIG. 3 shows an exemplary treatment system 300 for treating squamous cell carcinomas, basal cell carcinomas, and/or other cancers of the skin. Treatment system 300 may be configured to delivery energy to a cancerous lesion 331, which may be at or below a surface of the skin 330. Treatment system 300 may include a handpiece 310 that a user may use to direct energy delivery to a desired location. The handpiece 310 may include, for example, a handle 311 and/or a tip 312, which may be a disposable tip or may be an integral portion of the handpiece 310. Treatment system 300 may include an energy generator 355 that is operably connected to the handpiece 310 and generates the energy that is delivered to the cancerous lesion 331. In some embodiments, the energy generator 355 includes one or more lasers that generate laser light that is delivered to the handpiece 310 via one or more optical fibers 344. In some embodiments, the energy generator 355 generates electrical energy (e.g., radio frequency electrical energy) that is conveyed to one or more electrodes 342 of the handpiece 310 via one or more wires 340, with the electrodes being configured to deliver the electrical energy to the skin (e.g., through the tip 312, which may be electrically conductive or include electrically conductive regions). The energy generator 355 and handpiece 310 may be integrated in the same device or may be separate components connected by one or more cables.

The treatment system 300 may include one or more sensors 320. The sensor(s) 320 may be used, for example, to measure temperature, for visualization, and/or for another type of sensing. The sensor(s) 320 may include, for example, one or more infrared sensor(s), microwave sensor(s), cameras, and/or ultrasound sensor(s). In some embodiments, there are at least two sensors 320. One or more of the sensor(s) 320 may be a temperature sensor, such an infrared temperature sensor, used to monitor temperature of the skin 330 and of the cancerous lesion 331 before, during, and/or after treatment with lasers and/or radio frequency energy. One or more of the sensors 320 may be an ultrasound sensor(s) used to view the skin 330 and cancerous lesion 331 (e.g., below the surface of the skin) before, during, and/or after treatment, such as to determine the size (e.g., depth) of the lesion and/or the margins of the lesion before, during, and/or after treatment. The ultrasound sensor(s) may be used to view any changes in the cancerous lesion 331 from before, during, and/or after treatment. In some embodiments, at least one of the sensors 320 is a temperature sensor (e.g., an infrared temperature sensor) for sensing a temperature of the skin 330 and/or of the cancerous lesion 331 and at least one of the sensors is an ultrasound sensor for visualizing the skin 330 and/or of the cancerous lesion 331. In some embodiments, the sensor(s) 320 may be a temperature probe. The temperature probe may be placed in contact with or inserted into the skin 330 before, during, and/or after a treatment. The temperature probe may or may not be attached to the handpiece 310.

The one or more sensor(s) 320 may be located in different configurations in the treatment system 300. One or more of the sensor(s) 320 may be located (e.g., molded into, attached to, etc.) in the disposable tip 312 of the treatment system 300. The sensor(s) 320 may be located on or in the disposable tip 312 and/or on or in the handle 311. Sensor(s) 320 may be include at least one temperature sensor 322 and at least one ultrasound sensor 323 located in the disposable tip 312, any of which may be located (e.g., molded into, attached to, etc.) in the handle 311 and/or the disposable tip.

In some embodiments, data from the sensor(s) 320 (or data generated from signal generated by the sensor(s) 320) may be used to control operation of the treatment system 300. For example, data from the sensor(s) 320 may be used to control laser and/or radio frequency delivery to a treatment site. The sensor(s) 320 may be able to provide information such that the treatment system 300 may save information for future treatment (e.g., laser/radio frequency settings, temperature, images, information for each patient, clinical images, ultrasound images). The treatment system 300 may be able to save settings for both temperature and imaging (e.g., clinical, ultrasound) for each patient for future treatment.

The treatment system 300 may include a computing system 350 that may receive data from the sensor(s) 320. The computing system 350 may include one or more processors 351, one or more memories 352, and/or one or more displays 353. In some embodiments, the computing system 350 may receive data from the sensor(s) 320 and use the data to control operations of one or more energy generator(s) 355. In some embodiments, the computing system 350 may be configured to receive data from the sensor(s) 320 and store the data in the one or more memories 352 for future use, such as for post-treatment analysis. In some embodiments, the computing system 350 may display the data provided by the sensor(s) 320 or information generated from analysis of the data provided by the sensor(s) 320 on one or more displays 353. For example, a current temperature of the treatment site sensed by the sensor(s) 320 may be displayed on the one or more displays 353. In some embodiments, data from the one or more sensors 320 may be used to automatically control treatment. For example, the computing system 350 may compare a current temperature of the treatment site to one or more predefined thresholds and may start, stop, increase, and/or decrease the intensity of the energy delivery to the treatment site. For example, the computing system 350 may send control commands to energy generator 355 to start, stop, increase, and/or decrease energy delivery treatment. The energy generator 355 may be stopped and/or modulated (e.g., changing a pulse width and/or frequency to change the energy delivery) by the computing system 350, for example, when the computing system 350 determines that the temperature of a treatment site (e.g., the cancerous lesion 331) reaches a predetermined threshold (e.g., stored in memory 352) and/or reached a predetermined threshold for a predetermined time period associated with effective treatment. Optionally, the temperature may be monitored by the computing system 350 for a drop in temperature below a predetermined threshold (e.g., stored in memory 352) associated with effective treatment. For example, a treatment process controlled by the computing system 350 may include heating the treatment site at a predetermined temperature for a predetermined period of time. The amount of time that treatment is applied may be extended if the temperature of the treatment site drops below the predetermined temperature threshold as monitored by the computing system 350 using one or more sensors 320. This may include the computing system 350 starting a countdown timer once the treatment site reaches the predetermined temperature threshold, as determined by the computing system 350 based on the data from the one or more sensors 320, and pausing the countdown timer if the temperature of the treatment site drops below the predetermined temperature. The countdown timer may be restarted by the computing system 350 once the temperature of the treatment site rises above the predetermined temperature threshold. Optionally, treatment system 300 is used for treating a cancerous lesion without applying a topical medication. An assessment may be made whether treatment of the lesion with just system 300 was successful. If a determination is made that the treatment with just system 300 was successful (e.g., the lesion is gone), no topical medication may be used. If a determination is made that the treatment with just system 300 was not successful (e.g., at least some of the lesion remains), a topical medication may be used in accordance with the disclosure herein.

Accordingly, in some embodiments, provided herein is a method of treating skin cancer in an individual, the method comprising administering to a cancerous lesion on the individual a topical ointment for treating pre-cancerous or cancerous lesions in combination with energy from an energy delivery device. In some embodiments, the energy is a plurality of radio frequency pulses. In some embodiments, the energy is a plurality of laser pulses. In some embodiments, the plurality of laser pulses comprises a first plurality of passes using a first laser, and a second plurality of passes using a second laser. In some embodiments, each plurality of laser pulses comprises a plurality of passes using a laser, the plurality of passes comprising between 1 and 50 passes. In some embodiments, the first laser is an erbium laser and the second laser is a thulium laser. In some embodiments, the topical ointment is administered for between 2 and 10 days. In some embodiments, the topical medication is administered at least once per day. In some embodiments, the topical medication comprises tirbanibulin, imiquimod, or 5-fluorocil.

As described above, administration of a fractional laser and/or radio frequency therapy, to a cancerous lesion may enable topical medications to penetrate deeper into the skin and, as a result, may effectively inhibit the spread or cause destruction of cancerous cells at skin depths up to (and possibly exceeding) 2000 μm. Accordingly, in some embodiments, provided herein is a method of potentiating and/or improving the effects of a topical ointment for treatment of a cancerous lesion, the method comprising administering to a cancerous lesion energy from an energy delivery device. In some embodiments, the energy is a plurality of radio frequency pulses. In some embodiments, the energy is a plurality of laser pulses. In some embodiments, the plurality of laser pulses comprises a first plurality of passes using a first laser, and a second plurality of passes using a second laser. In some embodiments, each plurality of laser pulses comprises a plurality of passes using a laser, the plurality of passes comprising between 1 and 50 passes. In some embodiments, the first laser is an erbium laser and the second laser is a thulium laser. In some embodiments, the topical ointment is administered for between 2 and 10 days. In some embodiments, the topical medication is administered at least once per day. In some embodiments, the topical medication comprises tirbanibulin, imiquimod, or 5-fluorocil.

In one aspect, described herein is a method of treating skin cancer in an individual comprising administering to a cancerous lesion on the individual a topical ointment for treating pre-cancerous or cancerous lesions in combination with energy from an energy delivery device, wherein there is one or more synergistic therapeutic effects in the subject after administration relative to a baseline. In some embodiments, the one or more synergistic therapeutic effects is the size of the tumor derived from the cancer, the objective response rate, the duration of response, progression free survival, overall survival, or any combination thereof.

In some embodiments of the methods or uses or product for uses provided herein, response to administering to a cancerous lesion on the individual a topical ointment for treating pre-cancerous or cancerous lesions in combination with energy from an energy delivery device as described herein may include the RECIST Criteria 1.1. The RECIST Criteria 1.1 are as follows:

| | Category | Criteria |
|---|---|---|
| Based on | Complete | Disappearance of all target lesions. Any pathological |
| target lesions | Response (CR) | lymph nodes must have reduction in short axis to <10 |
| | | mm. |
| | Partial Response | ≥30% decrease in the sum of the longest diameter |
| | (PR) | (LD) of target lesions, taking as reference the baseline |
| | | sum of LDs. |
| | Stable Disease | Neither sufficient shrinkage to qualify for PR nor |
| | (SD) | sufficient increase to qualify for PD, taking as |
| | | reference the smallest sum of LDs while in trial. |
| | Progressive | ≥20% (and ≥5 mm) increase in the sum of the LDs of |
| | Disease (PD) | target lesions, taking as reference the smallest sum of |
| | | the target LDs recorded while in trial or the appearance |
| | | of one or more new lesions. |
| Based on non- | CR | Disappearance of all non-target lesions and |
| target lesions | | normalization of tumor marker level. All lymph nodes |
| | | must be non-pathological in size (<10 mm short axis). |
| | SD | Persistence of one or more non-target lesion(s) or/and |
| | | maintenance of tumor marker level above the normal |
| | | limits. |
| | PD | Appearance of one or more new lesions and/or |
| | | unequivocal progression of existing non-target lesions. |

In one embodiment of the methods or uses or product for uses provided herein, the effectiveness of treatment comprising administering to a cancerous lesion on the individual a topical ointment for treating pre-cancerous or cancerous lesions in combination with energy from an energy delivery device is assessed by measuring the objective response rate. In some embodiments, the objective response rate is the proportion of patients with tumor size reduction of a predefined amount and for a minimum period of time. In some embodiments the objective response rate is based upon RECIST v1.1. In one embodiment, the objective response rate is at least about 20%, at least about 25%, at least about 30%, at least about 35%, at least about 40%, at least about 45%, at least about 50%, at least about 60%, at least about 70%, or at least about 80%. In one embodiment, the objective response rate is at least about 20%-80%. In one embodiment, the objective response rate is at least about 30%-80%. In one embodiment, the objective response rate is at least about 40%-80%. In one embodiment, the objective response rate is at least about 50%-80%. In one embodiment, the objective response rate is at least about 60%-80%. In one embodiment, the objective response rate is at least about 70%-80%. In one embodiment, the objective response rate is at least about 80%. In one embodiment, the objective response rate is at least about 85%. In one embodiment, the objective response rate is at least about 90%. In one embodiment, the objective response rate is at least about 95%. In one embodiment, the objective response rate is at least about 98%. In one embodiment, the objective response rate is at least about 99%. In one embodiment, the objective response rate is at least 20%, at least 25%, at least 30%, at least 35%, at least 40%, at least 45%, at least 50%, at least 60%, at least 70%, or at least 80%. In one embodiment, the objective response rate is at least 20%-80%. In one embodiment, the objective response rate is at least 30%-80%. In one embodiment, the objective response rate is at least 40%-80%. In one embodiment, the objective response rate is at least 50%-80%. In one embodiment, the objective response rate is at least 60%-80%. In one embodiment, the objective response rate is at least 70%-80%. In one embodiment, the objective response rate is at least 80%. In one embodiment, the objective response rate is at least 85%. In one embodiment, the objective response rate is at least 90%. In one embodiment, the objective response rate is at least 95%. In one embodiment, the objective response rate is at least 98%. In one embodiment, the objective response rate is at least 99%. In one embodiment, the objective response rate is 100%.

In one embodiment of the methods or uses or product for uses provided herein, response to treatment comprising administering to a cancerous lesion on the individual a topical ointment for treating pre-cancerous or cancerous lesions in combination with energy from an energy delivery device is assessed by measuring the size of a tumor derived from the cancer (e.g., skin cancer). In one embodiment, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before treatment. In one embodiment, the size of a tumor derived from the cancer is reduced by at least about 10%-80%. In one embodiment, the size of a tumor derived from the cancer is reduced by at least about 20%-80%. In one embodiment, the size of a tumor derived from the cancer is reduced by at least about 30%-80%. In one embodiment, the size of a tumor derived from the cancer is reduced by at least about 40%-80%. In one embodiment, the size of a tumor derived from the cancer is reduced by at least about 50%-80%. In one embodiment, the size of a tumor derived from the cancer is reduced by at least about 60%-80%. In one embodiment, the size of a tumor derived from the cancer is reduced by at least about 70%-80%. In one embodiment, the size of a tumor derived from the cancer is reduced by at least about 80%. In one embodiment, the size of a tumor derived from the cancer is reduced by at least about 85%. In one embodiment, the size of a tumor derived from the cancer is reduced by at least about 90%. In one embodiment, the size of a tumor derived from the cancer is reduced by at least about 95%. In one embodiment, the size of a tumor derived from the cancer is reduced by at least about 98%. In one embodiment, the size of a tumor derived from the cancer is reduced by at least about 99%. In one embodiment, the size of a tumor derived from the cancer is reduced by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before treatment.

In one embodiment of the methods or uses or product for uses described herein, response to treatment comprising administering to a cancerous lesion on the individual a topical ointment for treating pre-cancerous or cancerous lesions in combination with energy from an energy delivery device as described herein is assessed by measuring the time of progression free survival after administering to a cancerous lesion on the individual a topical ointment for treating pre-cancerous or cancerous lesions in combination with energy from an energy delivery device. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after treatment.

In one embodiment of the methods or uses or product for uses described herein, response to treatment comprising administering to a cancerous lesion on the individual a topical ointment for treating pre-cancerous or cancerous lesions in combination with energy from an energy delivery device as described herein is assessed by measuring the time of overall survival after treatment. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after treatment.

### III. Patient Populations

The methods provided herein are useful for treatment of a range of individuals having skin cancer. For example, in some embodiments, the skin cancer is a basal cell skin cancer (BCC). In some embodiments, the BCC is nodular. In some embodiments, the BCC is infiltrative. In some embodiments, the BCC is superficial. In some embodiments, the skin cancer is a squamous cell skin cancers (SCC). In some embodiments, the SCC is in situ. Grading and/or staging of such cancers can be conducted, for example, according to the guidelines of the American Cancer Society.

In some embodiments, the BCC is high risk. Thus, in some embodiments, the BCC comprises one or more of the following features: a tumor on the trunk (chest or back), arm, or leg (other than the front of the lower leg) that is at least 2 cm across; a tumor on any other part of the body (head, neck, hands, feet, or genital area), regardless of size; a tumor lacking well-defined borders; a recurrent tumor; a tumor in a place that was previously treated with radiation; a tumor with an aggressive growth pattern when observed under a microscope; a tumor that has undergone perineural invasion; and the person with BCC has a weakened immune system. In some embodiments, the BCC is low risk (i.e., lacking all of the above features).

Accordingly, in some aspects, provided herein is a method of treating an individual having BCC, the method comprising administering to a cancerous lesion on the individual a topical ointment for treating pre-cancerous or cancerous lesions in combination with energy from an energy delivery device. In some embodiments, the energy is a plurality of radio frequency pulses. In some embodiments, the energy is a plurality of laser pulses. In some embodiments, the plurality of laser pulses comprises a first plurality of passes using a first laser, and a second plurality of passes using a second laser. In some embodiments, each plurality of laser pulses comprises a plurality of passes using a laser, the plurality of passes comprising between 1 and 50 passes. In some embodiments, the first laser is an erbium laser and the second laser is a thulium laser. In some embodiments, the topical ointment is administered for between 2 and 10 days. In some embodiments, the topical medication is administered at least once per day. In some embodiments, the topical medication comprises tirbanibulin, imiquimod, or 5-fluorocil.

In some embodiments, the SCC is very high risk. Thus, in some embodiments, the SCC comprises one or more of the following features: a tumor at least 4 cm across; cancer cells poorly differentiated (very abnormal) under a microscope; desmoplastic SCC; a tumor that is more than 6 mm deep, or it has grown beyond the fat layer below the skin (subcutaneous fat); cancer cells that have invaded a nerve deeper than the dermis layer of the skin; and cancer cells have invaded a blood vessel or lymph vessel in or near the tumor.

In some embodiments, the SCC is high risk. In other words, the SCC lacks all features of a very high risk SCC, but comprises one or more of the following features: a tumor on the trunk (chest or back), arm, or leg (other than the front of the lower leg), that is more than 2 cm but no more than 4 cm across; a tumor on any other part of the body (head, neck, hands, feet, front of the lower leg, or genital area), regardless of size; the extent of the tumor isn't well defined; a recurrent tumor; a tumor in a place that was previously treated with radiation, or in a place where there has been chronic inflammation; a tumor growing quickly; a tumor causing neurologic symptoms, such as pain or itching; a cancer is labeled as acantholytic, adenosquamous, or metaplastic SCC; a tumor 2 to 6 mm deep; a tumor that has undergone perineural invasion; and the person with SCC has a weakened immune system.

In some aspects, provided herein is a method of treating an individual having SCC, the method comprising administering to a cancerous lesion on the individual a topical ointment for treating pre-cancerous or cancerous lesions in combination with energy from an energy delivery device. In some embodiments, the energy is a plurality of radio frequency pulses. In some embodiments, the energy is a plurality of laser pulses. In some embodiments, the plurality of laser pulses comprises a first plurality of passes using a first laser, and a second plurality of passes using a second laser. In some embodiments, each plurality of laser pulses comprises a plurality of passes using a laser, the plurality of passes comprising between 1 and 50 passes. In some embodiments, the first laser is an erbium laser and the second laser is a thulium laser. In some embodiments, the topical ointment is administered for between 2 and 10 days. In some embodiments, the topical medication is administered at least once per day. In some embodiments, the topical medication comprises tirbanibulin, imiquimod, or 5-fluorocil.

In some embodiments, the skin cancer has a thickness of about 0.1 mm to about 1.5 mm, such as any of about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, or about 1.5 mm.

The methods of the present invention also provide important and significant treatment benefits compared to standard invasive treatments that may leave cosmetic blemishes on the skin. In some embodiments, the individual is ineligible for and/or is refusing an invasive treatment of skin cancer. Invasive treatment of skin cancers includes, for example, electrodesiccation and curettage (EDC) or cold steel surgery.

Accordingly, in some embodiments, provided herein is a method of treating skin cancer in an individual who is ineligible for and/or is refusing an invasive treatment of skin cancer, the method comprising administering to a cancerous lesion on the individual a topical ointment for treating pre-cancerous or cancerous lesions in combination with energy from an energy delivery device. In some embodiments, the energy is a plurality of radio frequency pulses. In some embodiments, the energy is a plurality of laser pulses. In some embodiments, the plurality of laser pulses comprises a first plurality of passes using a first laser, and a second plurality of passes using a second laser. In some embodiments, each plurality of laser pulses comprises a plurality of passes using a laser, the plurality of passes comprising between 1 and 50 passes. In some embodiments, the first laser is an erbium laser and the second laser is a thulium laser. In some embodiments, the topical ointment is administered for between 2 and 10 days. In some embodiments, the topical medication is administered at least once per day. In some embodiments, the topical medication comprises tirbanibulin, imiquimod, or 5-fluorocil.

In some embodiments, provided herein is a non-invasive method of treating skin cancer in an individual, the method comprising administering to a cancerous lesion on the individual a topical ointment for treating pre-cancerous or cancerous lesions in combination with energy from an energy delivery device. In some embodiments, the energy is a plurality of radio frequency pulses. In some embodiments, the energy is a plurality of laser pulses. In some embodiments, the plurality of laser pulses comprises a first plurality of passes using a first laser, and a second plurality of passes using a second laser. In some embodiments, each plurality of laser pulses comprises a plurality of passes using a laser, the plurality of passes comprising between 1 and 50 passes. In some embodiments, the first laser is an erbium laser and the second laser is a thulium laser. In some embodiments, the topical ointment is administered for between 2 and 10 days. In some embodiments, the topical medication is administered at least once per day. In some embodiments, the topical medication comprises tirbanibulin, imiquimod, or 5-fluorocil.

In some embodiments, also provided herein is a method of treating skin cancer in an individual ineligible for and/or is refusing external radiation therapy for skin cancer, the method comprising administering to a cancerous lesion on the individual a topical ointment for treating pre-cancerous or cancerous lesions in combination with energy from an energy delivery device. In some embodiments, the energy is a plurality of radio frequency pulses. In some embodiments, the energy is a plurality of laser pulses. In some embodiments, the plurality of laser pulses comprises a first plurality of passes using a first laser, and a second plurality of passes using a second laser. In some embodiments, each plurality of laser pulses comprises a plurality of passes using a laser, the plurality of passes comprising between 1 and 50 passes. In some embodiments, the first laser is an erbium laser and the second laser is a thulium laser. In some embodiments, the topical ointment is administered for between 2 and 10 days. In some embodiments, the topical medication is administered at least once per day. In some embodiments, the topical medication comprises tirbanibulin, imiquimod, or 5-fluorocil.

In some embodiments, the individual is human.

In one aspect, provided herein is a method of treating skin cancer in an individual, the method comprising administering to a cancerous lesion on the individual tirbanibulin once a day for five days, in combination with 12 passes of a 1550 nm erbium laser having a pulse energy of 55 mJ, followed by 12 passes of a 1927 nm thulium laser having pulse energy of 10 mJ.

### EMBODIMENTS

1. A method for treating squamous cancers of the skin, the method comprising:
administering a plurality of laser pulses to a cancerous lesion using one or more lasers; and
for a predetermined treatment period, repeatedly applying a topical medication for treating pre-cancerous or cancerous lesions to the cancerous lesion.
   2. The method of embodiment 1, wherein administering the plurality of laser pulses comprises:
administering a first plurality of laser pulses over a first plurality of passes using a first laser, and
administering a second plurality of laser pulses over a second plurality of passes using a second laser.
   3. The method of embodiment 2, wherein the first plurality of passes comprises between 1 and 50 passes.
   4. The method of embodiment 2, wherein the second plurality of passes comprises between 1 and 50 passes.
   5. The method of embodiment 2, wherein the first laser is an erbium laser and the second laser is a thulium laser.
   6. The method of embodiment 2, wherein a wavelength of the first laser is between 1500 nm and 1575 nm.
   7. The method of embodiment 2, wherein a pulse energy of the first laser is between 50 and 60 mJ.
   8. The method of embodiment 2, wherein a wavelength of the second laser is between 1875 nm and 1950 nm.
   9. The method of embodiment 2, wherein a pulse energy of the second laser is between 5 and 15 mJ.
   10. The method of embodiment 2, wherein an interval between each pass of the first plurality of passes is less than 3 seconds.
   11. The method of embodiment 2, wherein an interval between each pass of the second plurality of passes is less than 3 seconds.
   12. The method of embodiment 1, wherein the predetermined treatment period is between 2 and 10 days.
   13. The method of embodiment 1, wherein repeatedly applying the topical medication for treating pre-cancerous or cancerous lesions to the cancerous lesion comprises applying the topical medication at least once per day during the predetermined treatment period.
   14. The method of embodiment 1, wherein repeatedly applying the topical medication for treating pre-cancerous or cancerous lesions to the cancerous lesion comprises applying the topical medication to a region surrounding the cancerous lesion.
   15. The method of embodiment 14, wherein the region surrounding the cancerous lesion extends past a border of the cancerous lesion by at least 5 mm.
   16. The method of embodiment 15, wherein the region surrounding the cancerous lesion extends past the border of the cancerous lesion by at least 30 mm.
   17. The method of embodiment 1, wherein the topical medication comprises tirbanibulin.
   18. The method of embodiment 1, wherein the topical medication comprises imiquimod.
   19. The method of embodiment 1, wherein the topical medication comprises 5-fluorocil.
   20. The method of embodiment 1, wherein the cancerous lesion is located on the skin.
   21. The method of embodiment 1, comprising monitoring treatment with one or more sensors.
   22. The method of embodiment 21, wherein the one or more sensors comprises a temperature probe, an ultrasound sensor, an infrared sensor, or a microwave sensor.
   23. The method of embodiment 21, wherein at least one of the one or more sensors is disposed in a disposable tip of a handpiece used for administering the plurality of laser pulses.
   24. The method of embodiment 21, wherein at least one of the one or more sensors is disposed in a handpiece used for administering the plurality of laser pulses.
   25. The method of embodiment 21, wherein data from the one or more sensors is used to control the administration of the laser pulses.
   26. A method for treating cancers of the skin, the method comprising:
administering a plurality of radio frequency pulses to a cancerous lesion using one or more radio frequency emitting devices; and
for a predetermined treatment period, repeatedly applying a topical medication for treating pre-cancerous or cancerous lesions to the cancerous lesion.
   27. The method of embodiment 26, wherein the predetermined treatment period is between 2 and 10 days.
   28. The method of embodiment 26, wherein repeatedly applying the topical medication for treating pre-cancerous or cancerous lesions to the cancerous lesion comprises applying the topical medication at least once per day during the predetermined treatment period.
   29. The method of embodiment 26, wherein repeatedly applying the topical medication for treating pre-cancerous or cancerous lesions to the cancerous lesion comprises applying the topical medication to a region surrounding the cancerous lesion.
   30. The method of embodiment 29, wherein the region surrounding the cancerous lesion extends past a border of the cancerous lesion by at least 5 mm.
   31. The method of embodiment 29, wherein the region surrounding the cancerous lesion extends past the border of the cancerous lesion by at least 30 mm.
   32. The method of embodiment 26, wherein the topical medication comprises tirbanibulin.
   33. The method of embodiment 26, wherein the topical medication comprises imiquimod.
   34. The method of embodiment 26, wherein the topical medication comprises 5-fluorocil.
   35. The method of embodiment 26, wherein the cancerous lesion is located on the skin.
   36. The method of embodiment 26, comprising monitoring treatment with one or more sensors.
   37. The method of embodiment 36, wherein the one or more sensors comprises a temperature probe, an ultrasound sensor, an infrared sensor, or a microwave sensor.
   38. The method of embodiment 36, wherein at least one of the one or more sensors is disposed in a disposable tip.
   39. The method of embodiment 36, wherein at least one of the one or more sensors is disposed in a hand piece.
   40. The method of embodiment 36, wherein the information from the one or more sensors is used to control the administration of the radio frequency pulses.
   41. A method for treating squamous cancers of the skin, the method comprising:
administering a plurality of laser pulses to a squamous cancer of the skin using one or more lasers pulses having a wavelength in a range from 1000 nm to 10,600 nm.
   42. A method of treating skin cancer in an individual, the method comprising administering to a cancerous lesion on the individual a topical ointment in combination with energy from an energy delivery device.
   43. A method of treating skin cancer in an individual, the method comprising administering to a cancerous lesion on the individual energy from an energy delivery device in combination with a topical ointment.
   44. A method of potentiating and/or improving the effects of a topical ointment for treatment of a cancerous lesion, the method comprising administering to a cancerous lesion energy from an energy delivery device.
   45. A method of treating skin cancer in an individual who is ineligible for and/or is refusing an invasive treatment of skin cancer, the method comprising administering to a cancerous lesion on the individual a topical ointment for treating pre-cancerous or cancerous lesions in combination with energy from an energy delivery device.
   46. A system for treating cancers of the skin, the system comprising:
at least one energy generator configured to deliver energy to a cancerous lesion in the skin at a treatment site;
at least one sensor configured to sense a temperature of the treatment site;
a control system configured to control the at least one energy generator based on the temperature of the treatment site.
   47. The system of embodiment 46, comprising at least one sensor for visualizing the cancerous lesion.
   48. The system of embodiment 47, wherein at least one sensor for visualizing the cancerous lesion comprises an ultrasound sensor.
   49. The system of embodiment 46, comprising a handpiece, wherein the at least one energy generator and the at least one sensor are integrated into the handpiece.
   50. The system of embodiment 49, comprising at least one sensor for visualizing the cancerous lesion, wherein the at least one sensor is integrated into the handpiece.
   51. The system of embodiment 46, wherein the control system is configured to determine whether the treatment site has reached a predetermined temperature and to control the at least one energy generator to maintain the predetermined temperature for a predetermined time period.
   52. The system of embodiment 46, wherein the at least one energy generator is configured to deliver laser energy or electrical energy to the treatment site.

### EXAMPLE

An example study was performed to evaluate the combined effectiveness of fractional laser therapy and tirbanibulin ointment in treating squamous and basal cell carcinomas. The subjects of the study included ten men and two women aged 51-89 years with a mean age of 75 years with Fitzpatrick skin types ranging from 1 to 4. Subjects chosen had biopsy proven squamous or basal cell carcinomas. Overall, fifteen skin cancers including sites at the forehead, earlobe, scalp, nose, zygomatic arch, costal margin, mid back, flank, arm, and lower leg were treated (Table 1).

TABLE 1
Subject and Biopsy Data

| | | | | Approximate |
|---|---|---|---|---|
| | Fitzpatrick | Cancer | | Cancer Thickness |
| Subject | Scale | Type | Site | in Biopsy |
| A. Male, 63 y | 1 | Nodular BCCs | 1. Left | 1. Extends to base; |
| | | | lateral | at least 0.7 mm |
| | | | upper | 2. Extends to base; |
| | | | forehead | at least 0.45 mm |
| | | | 2. Right | 3. Extends to base; |
| | | | lateral mid | at least 0.6 mm |
| | | | to upper | |
| | | | forehead | |
| | | | 3. Right | |
| | | | costal | |
| | | | margin | |
| B. Male, 85 y | 3 | SCC in situ | Left lateral | 0.15 mm within the |
| | | | mid back | epidermis |
| C. Male, 88 y | 2 | Nodular & | Lt upper arm | Extends to base; at |
| | | infiltrative | | least 1.0 mm |
| | | BCC | | |
| D. Female, 51 y | 2 | Superficial | Anterior | 0.2 mm |
| | | BCC | mid to upper | |
| | | | left lower | |
| | | | leg at | |
| | | | midline | |
| E. Female, 80 y | 1 | SCC in situ | Right lateral | Unknown |
| | | | nose near | |
| | | | eyelid | |
| F. Male, 81 y | 3 | SCC in situ | Just above | Extends to base; at |
| | | | the left | least 0.1 mm |
| | | | zygomatic | |
| | | | arch | |
| G. Male, 62 y | 3 | Superficial & | Right | Extends to base; at |
| | | nodular BCC | medial | least 0.3 mm |
| | | | forearm | |
| H. Male, 64 y | 2 | Hypertrophic | Right | Extends to base, at |
| | | AK cannot | temple | least 0.4 mm |
| | | rule out SCC | | |
| I. Male, 89 y | 4 | SCC in situ | Left frontal | 1.10 mm |
| | | | parietal | |
| | | | scalp at | |
| | | | medial | |
| | | | hairline | |
| J. Male, 85 y | 2 | 1. Nodular | 1. Left nasal | Unknown |
| | | BCC | ala | |
| | | 2. Superficia | 2. Right | |
| | | 1 BCC | flank just | |
| | | | below axilla | |
| K. Male, 65 y | 2 | Nodular | Left mid | At least 1.1 mm, |
| | | BCC | paraspinal | extends to base |
| | | | back | |
| L. Male, 87 y | 1 | Invasive | Right | Extends to base, at |
| | | BCC & SCC | earlobe | least 0.3 mm |

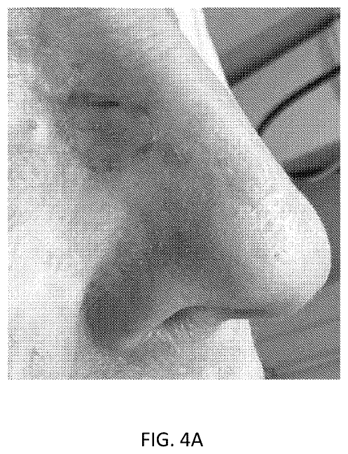

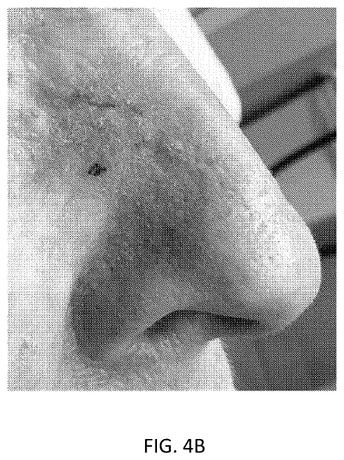

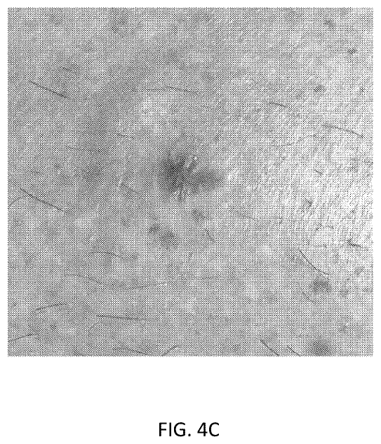

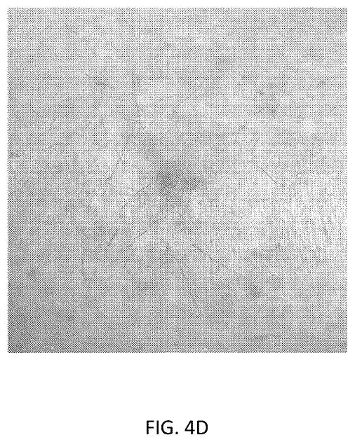

The site of the previously biopsied skin for each subject was disinfected with 70% isopropyl rubbing alcohol and injected prior to treatment with lidocaine with epinephrine. Lesions were first treated with the 1550 nm erbium laser (pulse energy of 55 mJ, treatment level of 8, and 12 passes) followed by the 1927 nm thulium laser (pulse energy of 10 mJ, treatment level of 8, and 12 passes). FIGS. 4A and 4B show immediately before and immediately after the fractional laser treatment on Subject E (Table 1). Pictures were taken before the fractional laser treatment and at each subsequent follow-up visit for a majority of patients.

Following the fractional laser treatment, participants were instructed on the proper use of tirbanibulin ointment using a sample as demonstration. Application instructions were followed in accordance with the product label. However, instead of only using tirbanibulin on the face/scalp as directed, subjects were instructed to apply the ointment to the biopsy-confirmed skin cancer sites that we treated with the fractional laser. Patients were called by phone during the five-night tirbanibulin application period to check progress.

No clinical recurrence of carcinoma was found in any of the participating patients as of ten months from the first subject treated. FIGS. 4C and 4D demonstrate clinical clearance of a superficial BCC on the lower leg of a Subject D (Table 1). This subject experienced mild post inflammatory hypopigmentation which is expected to resolve. Six sites were biopsied post-treatment. Histologically, no evidence of carcinoma was found in any sites. The sites on Subject B and E (Table 1) were biopsied at approximately five months and two months respectively post treatment. Both were SCCs in situ prior to treatment. The pathology reports showed no evidence of carcinoma at the re-biopsied sites. All three sites of Subject A (Table 1), formally nodular BCCS, were biopsied at seven months post-treatment for the forehead sites and six months post-treatment for the costal margin site. No evidence of carcinoma was found. Subject C had a BCC with nodular and infiltrative features that we biopsied 4.5 months post-treatment. No evidence of carcinoma was found. Results are pending for the six most recent participants who will be assessed at their upcoming follow-up visits.

The trial discussed in this example was designed to maximize the depth of skin penetration in order to effectively destroy cancerous cells while simultaneously providing the best cosmetic results. Bulk heating was a key method in maximizing the treatment region. The energy emitted by the fractional lasers can destroy keratinocytes, collagen fibers, and cancerous cells within the MTZ, but is also capable of affecting areas outside of the primary treatment zone of cell death. This is termed heat shock response. This response increases with an increase in temperature. As skin temperature increases, the diameter of the MTZ has been found to increase. At a pulse energy of around 40 mJ, the depth of the resulting MTZ can be over 1000 μm and the width at almost 200 μm. Increasing pulse energy correlates with increasing MTZ depth and width, all without harming the healthy surrounding tissue or damaging its structure.

Users of the fractional lasers often utilize a cooling system during cosmetic treatment to increase comfort during the procedure for patients. However, since the affected area was numbed prior to treatment, cooling was not needed for pain management, and therefore results in more bulk heating. Additionally, by limiting interval time between passes of the fractional laser to less than 2-3 seconds, the ability of heat to diffuse through the epidermis, dermis, and through blood perfusion, was limited. Finally, we predict that the MTZ created by fractional laser therapy allows the tirbanibulin ointment to penetrate deeper into the skin resulting in greater skin cancer destruction and preventing recurrence. The application of tirbanibulin ointment also creates a field effect of pre-cancerous cell destruction, as the subjects are instructed to apply approximately 25 mm in diameter around the lesion.

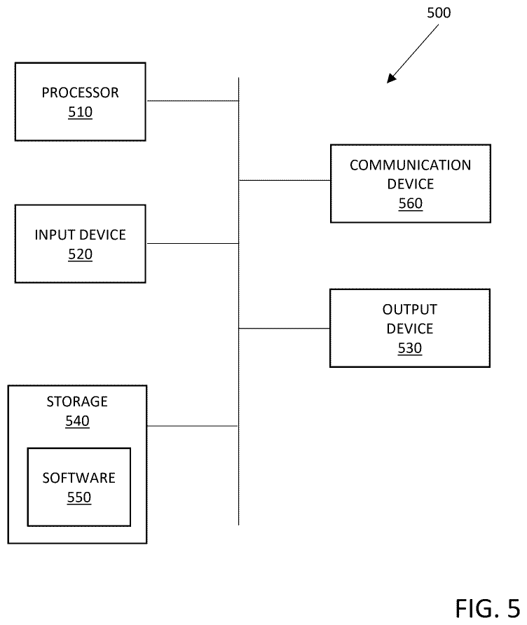

FIG. 5 illustrates an example of a computing system 500 that can be used for any computing systems described herein, such as for a computing system controlling energy delivery device 104 of FIG. 1 and/or for computing system 350 of FIG. 3. System 500 can be a computer connected to a network, such as one or more networks of a hospital, including a local area network within a room of a medical facility and a network linking different portions of the medical facility. System 500 can be a client or a server. System 500 can be any suitable type of processor-based system, such as a personal computer, workstation, server, handheld computing device (portable electronic device) such as a phone or tablet, or dedicated device. System 500 can include, for example, one or more of input device 520, output device 530, one or more processors 510, storage 540, and communication device 560. Input device 520 and output device 530 can generally correspond to those described above and can either be connectable or integrated with the computer.

Input device 520 can be any suitable device that provides input, such as a touch screen, keyboard or keypad, mouse, gesture recognition component of a virtual/augmented reality system, or voice-recognition device. Output device 530 can be or include any suitable device that provides output, such as a display, touch screen, haptics device, virtual/augmented reality display, or speaker.

Storage 540 can be any suitable device that provides storage, such as an electrical, magnetic, or optical memory including a RAM, cache, hard drive, removable storage disk, or other non-transitory computer readable medium. Communication device 560 can include any suitable device capable of transmitting and receiving signals over a network, such as a network interface chip or device. The components of the computing system 500 can be connected in any suitable manner, such as via a physical bus or wirelessly.

Processor(s) 510 can be any suitable processor or combination of processors, including any of, or any combination of, a central processing unit (CPU), field programmable gate array (FPGA), graphics processing unit (GPU), and application-specific integrated circuit (ASIC). Software 550, which can be stored in storage 540 and executed by one or more processors 510, can include, for example, the programming that embodies the functionality or portions of the functionality of the present disclosure (e.g., as embodied in the devices as described above), such as programming for performing step 1 of FIG. 1 and/or step 204 of method 200.

Software 550 can also be stored and/or transported within any non-transitory computer readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 540, that can contain or store programming for use by or in connection with an instruction execution system, apparatus, or device.

Software 550 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport computer readable medium can include, but is not limited to, an electronic, magnetic, optical, electromagnetic, or infrared wired or wireless propagation medium.

System 500 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, Ethernet cables, fiber optic cables, DSL, or telephone lines.

System 500 can implement any operating system suitable for operating on the network. Software 550 can be written in any suitable programming language, such as C, C++, Java, or Python. In various examples, application software embodying the functionality of the present disclosure can be deployed in different configurations, such as in a client/server arrangement or through a web browser as a web-based application or web service, for example.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A method for treating squamous cancers of the skin, the method comprising:
administering a plurality of laser pulses to a cancerous lesion using one or more lasers; and
for a predetermined treatment period, repeatedly applying a topical medication for treating pre-cancerous or cancerous lesions to the cancerous lesion.

2. The method of claim 1, wherein administering the plurality of laser pulses comprises:
administering a first plurality of laser pulses over a first plurality of passes using a first laser, and
administering a second plurality of laser pulses over a second plurality of passes using a second laser.

3. The method of claim 2, wherein the first plurality of passes comprises between 1 and 50 passes.

4. The method of claim 2, wherein the second plurality of passes comprises between 1 and 50 passes.

5. The method of claim 2, wherein the first laser is an erbium laser and the second laser is a thulium laser.

6. The method of claim 2, wherein a wavelength of the first laser is between 1500 nm and 1575 nm.

7. The method of claim 2, wherein a pulse energy of the first laser is between 50 and 60 mJ.

8. The method of claim 2, wherein a wavelength of the second laser is between 1875 nm and 1950 nm.

9. The method of claim 2, wherein a pulse energy of the second laser is between 5 and 15 mJ.

10. The method of claim 2, wherein an interval between each pass of the first plurality of passes is less than 3 seconds.

11. The method of claim 2, wherein an interval between each pass of the second plurality of passes is less than 3 seconds.

12. The method of claim 1, wherein the predetermined treatment period is between 2 and 10 days.

13. The method of claim 1, wherein repeatedly applying the topical medication for treating pre-cancerous or cancerous lesions to the cancerous lesion comprises applying the topical medication at least once per day during the predetermined treatment period.

14. The method of claim 1, wherein repeatedly applying the topical medication for treating pre-cancerous or cancerous lesions to the cancerous lesion comprises applying the topical medication to a region surrounding the cancerous lesion.

15. The method of claim 14, wherein the region surrounding the cancerous lesion extends past a border of the cancerous lesion by at least 5 mm.

16. The method of claim 15, wherein the region surrounding the cancerous lesion extends past the border of the cancerous lesion by at least 30 mm.

17. The method of claim 1, wherein the topical medication comprises tirbanibulin.

18. The method of claim 1, wherein the topical medication comprises imiquimod.

19. The method of claim 1, wherein the topical medication comprises 5-fluorocil.

20. The method of claim 1, wherein the cancerous lesion is located on the skin.

21. The method of claim 1, comprising monitoring treatment with one or more sensors.

22. The method of claim 21, wherein the one or more sensors comprises a temperature probe, an ultrasound sensor, an infrared sensor, or a microwave sensor.

23. The method of claim 21, wherein at least one of the one or more sensors is disposed in a disposable tip of a handpiece used for administering the plurality of laser pulses.

24. The method of claim 21, wherein at least one of the one or more sensors is disposed in a handpiece used for administering the plurality of laser pulses.

25. The method of claim 21, wherein data from the one or more sensors is used to control the administration of the laser pulses.

26. A method for treating cancers of the skin, the method comprising:
administering a plurality of radio frequency pulses to a cancerous lesion using one or more radio frequency emitting devices; and
for a predetermined treatment period, repeatedly applying a topical medication for treating pre-cancerous or cancerous lesions to the cancerous lesion.

27. The method of claim 26, wherein the predetermined treatment period is between 2 and 10 days.

28. The method of claim 26, wherein repeatedly applying the topical medication for treating pre-cancerous or cancerous lesions to the cancerous lesion comprises applying the topical medication at least once per day during the predetermined treatment period.

29. The method of claim 26, wherein repeatedly applying the topical medication for treating pre-cancerous or cancerous lesions to the cancerous lesion comprises applying the topical medication to a region surrounding the cancerous lesion.

30. The method of claim 29, wherein the region surrounding the cancerous lesion extends past a border of the cancerous lesion by at least 5 mm.

31. The method of claim 29, wherein the region surrounding the cancerous lesion extends past the border of the cancerous lesion by at least 30 mm.

32. The method of claim 26, wherein the topical medication comprises tirbanibulin.

33. The method of claim 26, wherein the topical medication comprises imiquimod.

34. The method of claim 26, wherein the topical medication comprises 5-fluorocil.

35. The method of claim 26, wherein the cancerous lesion is located on the skin.

36. The method of claim 26, comprising monitoring treatment with one or more sensors.

37. The method of claim 36, wherein the one or more sensors comprises a temperature probe, an ultrasound sensor, an infrared sensor, or a microwave sensor.

38. The method of claim 36, wherein at least one of the one or more sensors is disposed in a disposable tip.

39. The method of claim 36, wherein at least one of the one or more sensors is disposed in a hand piece.

40. The method of claim 36, wherein the information from the one or more sensors is used to control the administration of the radio frequency pulses.

41. A method for treating squamous cancers of the skin, the method comprising:
administering a plurality of laser pulses to a squamous cancer of the skin using one or more lasers pulses having a wavelength in a range from 1000 nm to 10,600 nm.

42. A system for treating cancers of the skin, the system comprising:
at least one energy generator configured to deliver energy to a cancerous lesion in the skin at a treatment site;
at least one sensor configured to sense a temperature of the treatment site;
a control system configured to control the at least one energy generator based on the temperature of the treatment site.

43. The system of claim 42, comprising at least one sensor for visualizing the cancerous lesion.

44. The system of claim 43, wherein at least one sensor for visualizing the cancerous lesion comprises an ultrasound sensor.

45. The system of claim 42, comprising a handpiece, wherein the at least one energy generator and the at least one sensor are integrated into the handpiece.

46. The system of claim 45, comprising at least one sensor for visualizing the cancerous lesion, wherein the at least one sensor is integrated into the handpiece.

47. The system of claim 42, wherein the control system is configured to determine whether the treatment site has reached a predetermined temperature and to control the at least one energy generator to maintain the predetermined temperature for a predetermined time period.

48. The system of claim 42, wherein the at least one energy generator is configured to deliver laser energy or electrical energy to the treatment site.
